(19) United States
(12) Patent Application Publication
(10) Pub. No.: US 2010/0034739 A1
(43) Pub. Date: Feb. 11, 2010

(54) Fungus-Specific Imaging Agents

(75) Inventors: Chun Li, Missouri City, TX (US); Dimitrios P. Kontoyiannis, Bellaire, TX (US)

Correspondence Address: BAKER BOTTS L.L.P.;PATENT DEPARTMENT, 98 SAN JACINTO BLVD., SUITE 1500, AUSTIN, TX, 78701-4039, US

(73) Assignee: BOARD OF REGENTS, THE UNIVERSITY OF TEXAS SYSTEM, Austin, TX (US)

(21) Appl. No.: 12/227,155

(22) PCT Filed: May 11, 2007
(86) PCT No.: PCT/US2007/068766

§ 371 Date: Nov. 7, 2008

Related U.S. Application Data
(60) Provisional application No. 60/747,044, filed on May 11, 2006.

(51) Int. Cl. A61K 51/00; A61K 49/00

(52) U.S. Cl. 424/1.69; 424/9.1

(57) ABSTRACT

The disclosure relates to an imaging composition including a fungus-specific peptide and an imaging material. Another imaging composition includes a fungus-specific peptide and a chelator able to chelate a radionuclide. The disclosure also provides to a method of detecting a fungal infection. The method includes administering an imaging agent to a patient. The imaging agent comprises a fungus-specific peptide and an imaging material. Then one may detect the imaging agent in the patient. Detecting retained imaging agent in a tissue or organ indicates fungal infection of the tissue or organ.

### RELATED APPLICATION

This application claims priority to U.S. Provisional Patent Application Ser. No. 60/747,044, filed May 11, 2006, and entitled “Fungus-Specific Imaging Agents.” the contents of which are incorporated herein in their entirety by reference.

### TECHNICAL FIELD

The present disclosure relates to fungus-specific imaging agents. In particular embodiments, it relates to radiolabeled peptides. These agents may be used for diagnosis or treatment of fungal infections, including Aspergillus and Rhizopus infections.

### BACKGROUND

Both Aspergillus and Rhizopus are able to infect mammals. Because fungi are more similar to mammalian cells than bacteria, these types of fungal infections are more difficult to treat than many bacterial infections. Thus earlier detection, when there is normally less fungus to kill, may lead to improved treatment results. Additionally, because fungal infections can be difficult to treat, additional forms of treatment are also beneficial.

Some infections by Aspergillus and Rhizopus are located on the skin. As a result, they may be diagnosed without significant difficulty and respond reasonably well to current treatments. Aspergillus and Rhizopus, however, may also infect areas that are difficult to access. Invasive aspergillosis is the most common opportunistic fungal infection. It is especially common in immunocompromised patients, such as patients with leukemia and transplant recipients. Patient outcomes are poor and invasive aspergillosis is often fatal, particularly for children, but outcomes are significantly improved with early detection and early administration of anti-fungal therapy. In particular, pulmonary invasive aspergillosis is a threat to patients, especially immunocompromised patients.

Currently, pulmonary invasive aspergillosis and pulmonary Rhizopus infection are diagnosed using chest X-rays and high-resolution chest computed tomography (CT). These methods are only able to provide anatomical information. They cannot specifically detect the fungus. Instead, they look for structural changes in the lungs, which are often absent in early stages of infection when treatment would be most beneficial. Further, the presence of scar tissue in the lungs may complicate anatomical diagnosis.

### SUMMARY

One embodiment of the disclosure relates to an imaging composition including a fungus-specific peptide and an imaging material. Another embodiment relates to an imaging composition including a fungus-specific peptide and a chelator able to chelate a radionuclide.

Other embodiments of the disclosure relates to a method of detecting a fungal infection. The method includes administering an imaging agent to a patient. The imaging agent comprises a fungus-specific peptide and an imaging material. Then one may detect the imaging agent in the patient. Detecting retained imaging agent in a tissue or organ indicates fungal infection of the tissue or organ.

### BRIEF DESCRIPTION OF THE DRAWINGS

A more complete understanding of the present disclosure thereof may be acquired by referring to the following description taken in conjunction with the accompanying drawings. These drawings represent only certain embodiments of the present disclosure.

FIG. 1 illustrates the structure of an ¹¹¹In-labeled peptide imaging agent, ¹¹¹In-DTPA-Benzyl-NH-Succinic Acid-CGGRLGPFC (SEQ. ID. NO:1) (also called “¹¹¹In-DTPA-SA-CGGRLGPFC”) targeted to aspergillosis.

FIG. 2 illustrates gamma scintography of mice injected with the ¹¹¹In-labeled peptide of FIG. 1. Control mice did not have a fungal infection, while infected mice had acute pulmonary aspergillosis. Arrows indicate radiotracer in the lung.

FIG. 3A illustrates the biodistribution of the ¹¹¹In-labeled peptide of FIG. 1 twenty-four (24) hours after injection in control mice without a fungal infection, or in mice infected with Aspergillus fumigatus. Data are expressed as mean +/− standard deviation (n=5).

FIG. 3B illustrates the target to background ratio of the ¹¹¹In-labeled peptide of FIG. 1 twenty-four (24) hours after injection in control mice without a fungal infection, or in mice infected with Aspergillus fumigatus. Data are presented as the ratio or percentage of injected dose per gram of tissue (n=5).

FIG. 4 illustrates the structure of an ⁶⁸Ga-labeled peptide imaging agent (called “⁶⁸Ga-DOTA-CGGRLGPFC”).

FIG. 5A illustrates μ-PET images of mice injected with the ⁶⁸Ga-labeled peptide of FIG. 4. Normal mice did not have a fungal infection, while infected mice had acute pulmonary aspergillosis. Arrows indicate accumulated imaging agent.

FIG. 5B illustrates autoradiography of the lungs of mice injected with the ⁶⁸Ga-labeled peptide of FIG. 4. Normal mice did not have a fungal infection, while infected mice had acute pulmonary aspergillosis.

FIG. 6 illustrates histology and autoradiography of excised lung tissue from a mouse injected with Ga-68-labeled peptide of FIG. 4. The mouse had acute pulmonary aspergillosis. Aspergillus was demonstrated with the Grocott methenamine-silver nitrate (GMS) fungus staining technique.

### DESCRIPTION

The present disclosure relates to fungus-specific imaging agents. In particular embodiments, it relates to radiolabeled peptides. These imaging agents may be used for diagnosis or treatment of fungal infections, including Aspergillus and Rhizopus infections.

A fungus-specific imaging agent of the present disclosure may include at least one fungus-specific peptide and at least one imaging material. In specific embodiments, it may also include a molecule for complexing the fungus-specific peptide and the imaging material. A general diagram of an example imaging agent is as follows:

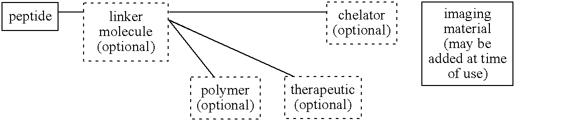

The imaging agents of the current disclosure may include the cyclic peptide c(CGGRLGPFC) (SEQ. ID. NO:1) or c(CWGHSRDEC) (SEQ. ID. NO:2) as the peptide. These peptides have been shown to bind in vitro to the hyphae of Aspergillus and Rhizopus. (Lionakis, M. S. et al., Development of a Ligand-Directed Approach to Study the invasive Aspergillosis, Infect. Immun. 73(11):7747-7758 (2005), incorporated by reference herein.) As described herein, these peptides may be used to form fungus-specific imaging agents of the current disclosure, which specifically detect fungal infection in vivo.

The imaging material may be any imaging material suitable for use with the type of diagnosis desired. In particular, for lungs it may be any imaging material compatible with lung diagnosis. For example, the imaging material may be a nuclear imaging material, such as a radionuclide. In some embodiments, the radionuclide may include ¹⁸F, ¹³¹I, ¹²⁴I, ¹²⁵I, ¹¹¹In, ^{99m}Tc, ⁶⁷Cu, ⁶⁴Cu, ⁶⁸Ga and/or combinations thereof.

In other exemplary embodiments, the imaging material may be an MRI imaging material. MRI imaging materials may generally include any paramagnetic imaging materials, including, but not limited to, paramagnetic imaging materials based on liposomes or nanoparticles. In other exemplary embodiments, the MRI imaging material may include Gd, Mn or iron oxide.

In other embodiments, other imaging materials known in the art may be used for a particular imaging technique.

Although single peptides are complexed with single imaging materials in many examples of this disclosure, other embodiments of the invention include single or multiple peptides (of the same or different types) complexed with single or multiple imagining materials (also of the same or different types) to form a single imaging agent.

The peptide may be complexed with the imaging material using any methods known in the art or later discovered, as modified with the benefit of this disclosure. In particular the peptide may be associated with a chelator, for example through a covalently bound linker molecule. The chelator may then chelate the imaging material, particularly a radionuclide.

Chelators which are often used to bind metal ions include but are not limited to:

diethylenetriaminepentaacetic acid (DTPA);

p-aminobenzyl-diethylenetriaminepentaacetic acid (p-NH₂-Bz-DTPA);

ethylene diaminetetracetic acid (EDTA);

1,4,7,10-tetraazacyclododecane-N,N′,N″,N′″-tetraacetic acid (DOTA);

2-p-aminobenzyl-1,4,7,10-tetraazacyclododecane-N,N′,N″,N′″-tetraaceticacid (p-NH₂-Bz-DOTA);

1,4,7,10-tetraazacyclododecane-1,4,7,10-tetrakis(methylene phosphonic acid) (DOPA); and

3,6,9,15-tetraazabicyclo[9.3.1]pentadeca-1(15),11,13-triene-3,6,9-triacetic acid (PCTA).

These chelators may be attached to the peptide using a linker molecule, for example succinic Acid, polyethylene glycol, lysine, an amino acid, an aliphatic chain and combination thereof. Some chelating agents may also be directly bound to the peptide. A tyrosine unit may be introduced to the peptide for radiolabeling with iodine isotopes.

Embodiments of the fungus-specific imaging agents of the present disclosure may additionally include larger polymers. These polymers make the imaging peptides larger, so that they are not absorbed by the body as quickly or are not filtered by the kidneys as quickly. Any biocompatible polymer may be used. Biocompatible polymers may include, for example, poly(L-Glutamic acid) other poly(amino acids), polyethylene glycol(PEG) and/or an aliphatic chain. The biocompatible polymer may be selected to have a size at above that of the glomerular filtration threshold of approximately 45 Å in hydrodynamic radius. In some embodiments, the polymer may be used in place of the linker molecule to connect the peptide and chelator or imaging material. It may also be bonded to either the peptide or the linker material.

The imaging material, particularly a radionuclide, may have a therapeutic as well as a diagnostic effect. Ionizing radiation delivered by specific antibody has been shown previously to be effective for therapeutic against fungal infection (Dadachova E et al, PNAS, 100: 10942-10947, 2003). However, in some embodiments, a therapeutic may additionally be attached to a fungus-specific imaging agent of the present disclosure. This may, for example, allow detection of where the therapeutic does not reach, which may be used to determine whether additional treatment is administered.

All imaging agents may be provided in a pharmaceutically acceptable carrier, including a carrier adapted to a particular form of administration, such as an aerosol, injectable formulation, or other liquid. Imaging agents may be stored as lyophilized powder or in concentrated form. Due to the short time period during which radionuclides are useful, all of the rest of the imaging agent may be provided, with the radionuclide added near the time of use. Imaging agents using a chelating agent may be particularly well-suited for addition of the imaging material by the user or otherwise near the time of use. Accordingly, some embodiments of the invention are directed to an imaging agent that contains all elements described above but the imaging material.

Methods of the current disclosure include detecting a fungal infection, particularly as Aspergillus or Rhizopus infection, in a mammal using a fungus-specific imaging agent as described above. The method may in particular include detection of infection in a internal bodily area, such as the lungs and respiratory pathways. These methods may be used to detect fungal infection at any stage, although, in exemplary embodiments it may be used to detect early-stage infection, particularly infection too early to be detected using anatomical methods such as chest X-rays or CT scans. The detection methods of the present disclosure may also be used to monitor fungal infection or the effects of treatment, in particular in patients with scarring that interferes with detection using anatomical methods. The detection methods may be used to detect actual fungus living in the patient in a fungus-specific manner.

Detection may include administering a fungus-specific imaging agent to a mammal, such as a human patient, then performing a medical scan able to detect the imaging material of the imaging agent. In specific embodiments, PET scans, gamma scintography, MRI's and other nuclear imaging may be used. In other embodiments optical imaging, such as near-infrared imaging may be used.

The imaging agent may be administered in any manner compatible with the type of detection, infected (or potentially infected) area, and patient. For example, it may be administered by inhalation or intravenous injection. Injected agents may be administered at a dose of approximately 4000 μCi/patient for gamma scintigraphy, or approximately 10,000 μCi/patient for PET imaging.

Detection may occur at any time during which the imaging material remains suitable for imaging. In particular, it may occur within thirty (30) and one hundred twenty (120) minutes after administration of the imaging agent. Because the fungus-specific imaging agent bind specifically to the hyphae of Aspergillus and Rhizopus, infection with either fungus, particularly acute pulmonary invasive aspergillosis, may be detected by accumulation of radioactive material in the area of infection. Using these methods, infection may be detectable even when it is not detectable using anatomical methods. Additionally, if a therapeutic is included in the fungus-specific imaging agent, areas that have not received the therapeutic may also be detected.

### EXAMPLES

The following examples provide details of certain embodiments of the invention, they are not intended to and should not be interpreted to disclose every feature of the invention as a whole.

### Example 1

### ¹¹¹In-Labeled Peptide Imaging Agent, Gamma Scintography, and Retention of Imaging Agent in Infected Lung

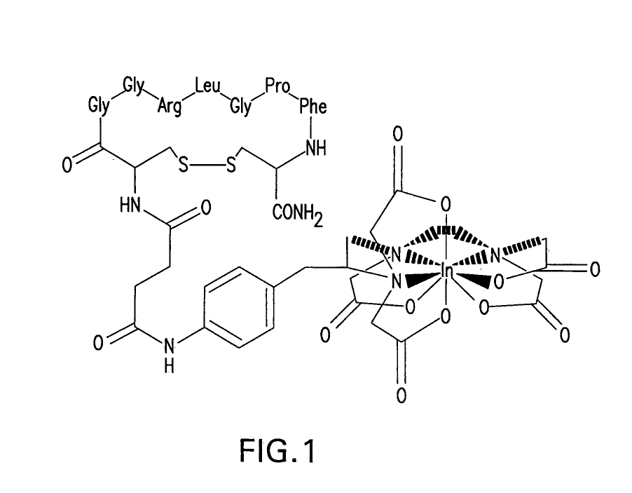

An imaging agent having the structure of FIG. 1 was synthesized. In addition to the peptide c(CGGRLGPFC) (SEQ. ID. NO:1), the imaging agent contains a Benzyl-NH-Succinic Acid linker molecule, a DTPA chelator and an ¹¹¹In imaging material.

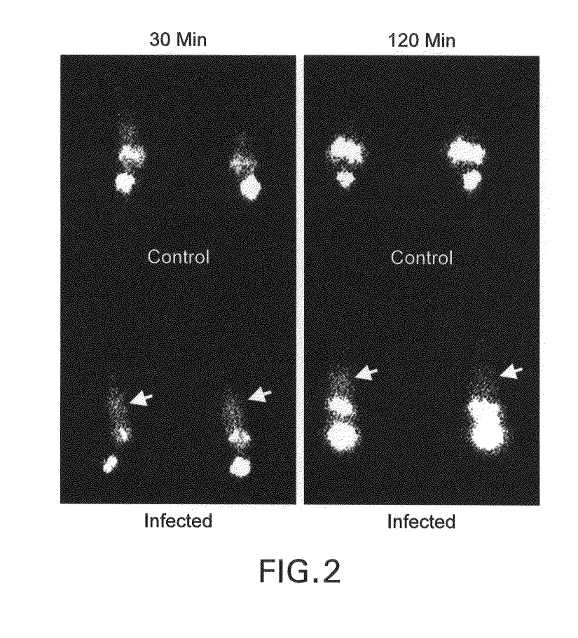

Mice weighing approximately 20 g each were injected intravenously with the imaging agent of FIG. 1 to provide radioisotope at a level of approximately 80 μCi per mouse. Gamma scintography was performed at 30 and 120 minutes after injection. Control mice had no fungal infection, while infected mice had acute pulmonary aspergillosis. Experiments were performed 1 to 2 days after infection. Gamma scintography images of control and infected mice are shown in FIG. 2. The same mouse is shown for each test type at 30 and 120 minutes. Arrows in FIG. 2 indicate the accumulated radiotracer. Radiotracer accumulation in the lungs of the control mouse was not visible at 120 minutes after injection. Increased radiotracer could be seen as little as 5 minutes after injection.

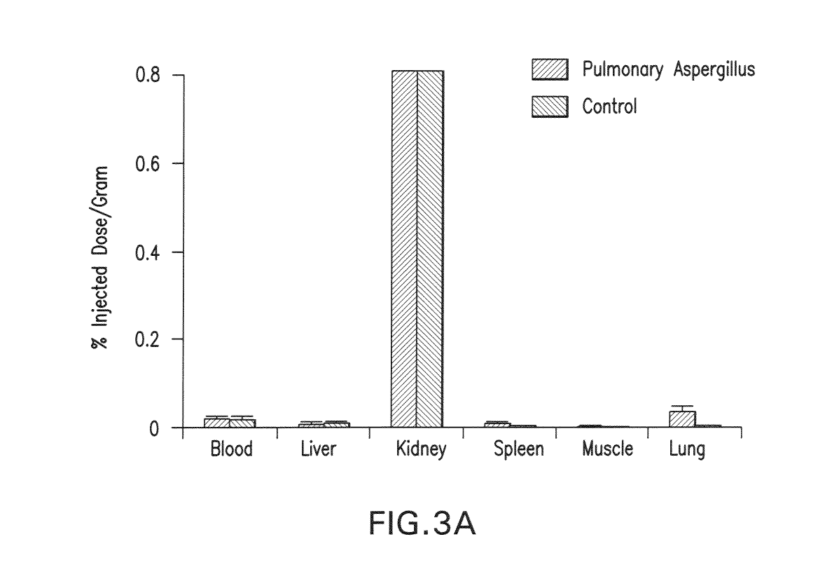

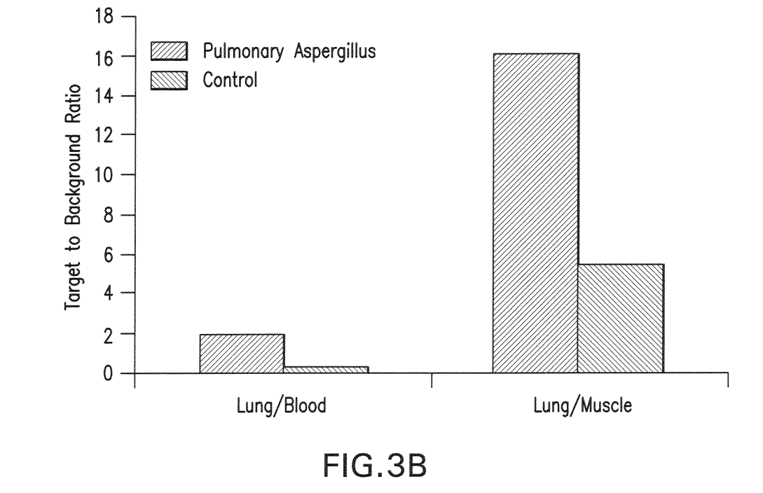

The biodistribution of the imaging agent was also evaluated 24 hours after injection. The results of this study are presented in FIG. 3A. In particular, higher uptake of the imaging agent was seen in the lung of the mice infected with Aspergillus fumigatus than in the uninfected control mice. Mice used in this experiment were the same as those shown in FIG. 2.

Target-to-background ratio at 24 hours post injection was also evaluated and the results are presented in FIG. 3B. Mice with a pulmonary Aspergillus infection showed much higher amounts of imaging agent in lung tissue as compared to blood or muscle than did control mice.

### Example 2

### ⁶⁸In-Labeled Peptide Imaging Agent, μ-PET Imaging, Evaluation of Radioactivity in Lung Sections

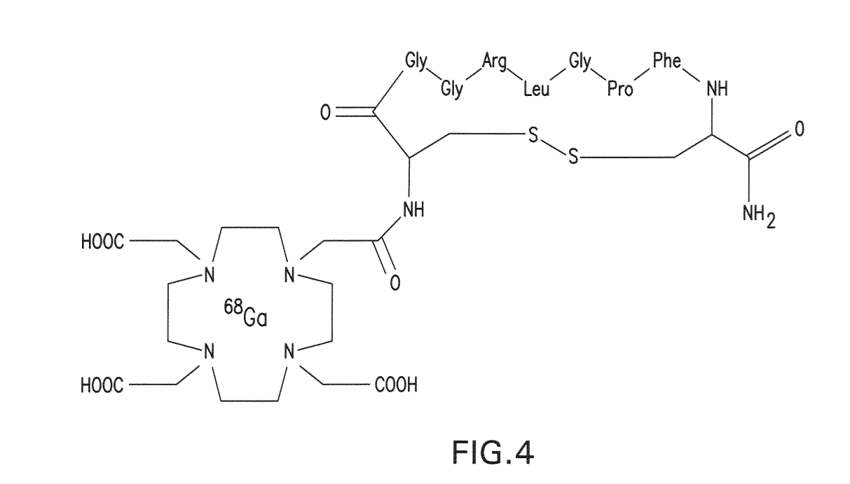

An imaging agent having the structure of FIG. 4 was synthesized. In addition to the peptide c(CGGRLGPFC) (SEQ. ID. NO:1), the imaging agent contains a DOTA chelator and an ⁶⁸Ga imaging material.

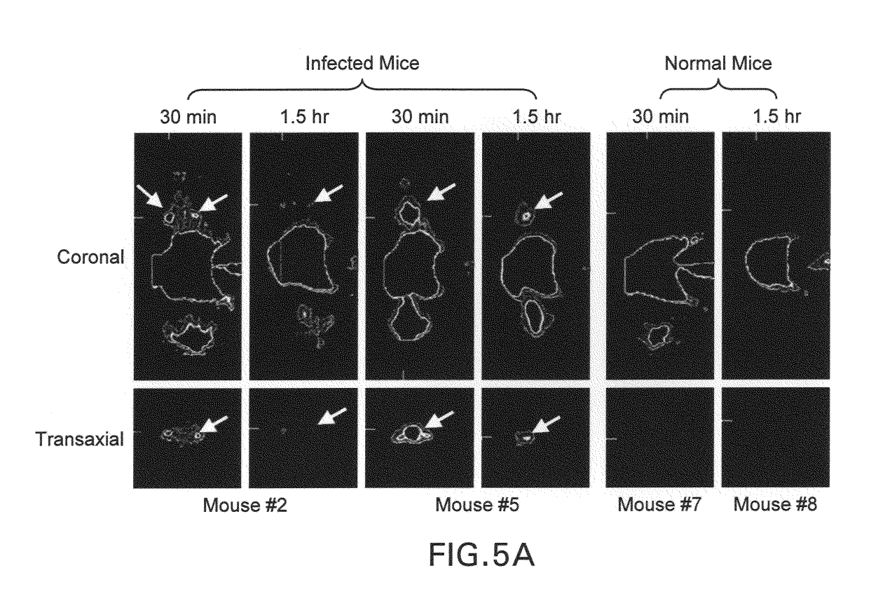

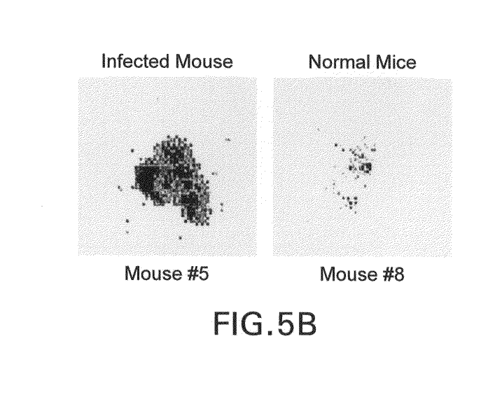

Mice weighing approximately 20 g each were injected intravenously with the imaging agent of FIG. 4 to provide radioisotope at a level of approximately 200 μCi per mouse. μ-PET imaging was performed at 30 and 90 minutes after injection. Control mice had no fungal infection, while infected mice had acute pulmonary aspergillosis. μ-PET images of normal and infected mice are shown in FIG. 5A. Arrows in FIG. 5A indicate the accumulated radiotracer. Radiotracer accumulation can be clearly seen in the lungs of the infected mice, but not the normal mice.

Lungs were removed from the mice after the 90 minute imaging session and snap frozen, then cut into 20 μm slices. These slices were air-dried and exposed to a phosphors screen. The screen was exposed for 10 minutes. Example results are shown in FIG. 5B. Heterogeneous distribution of radioactivity may be seen in the lungs of the infected mouse. Little radioactivity is seen in the normal mouse lungs.

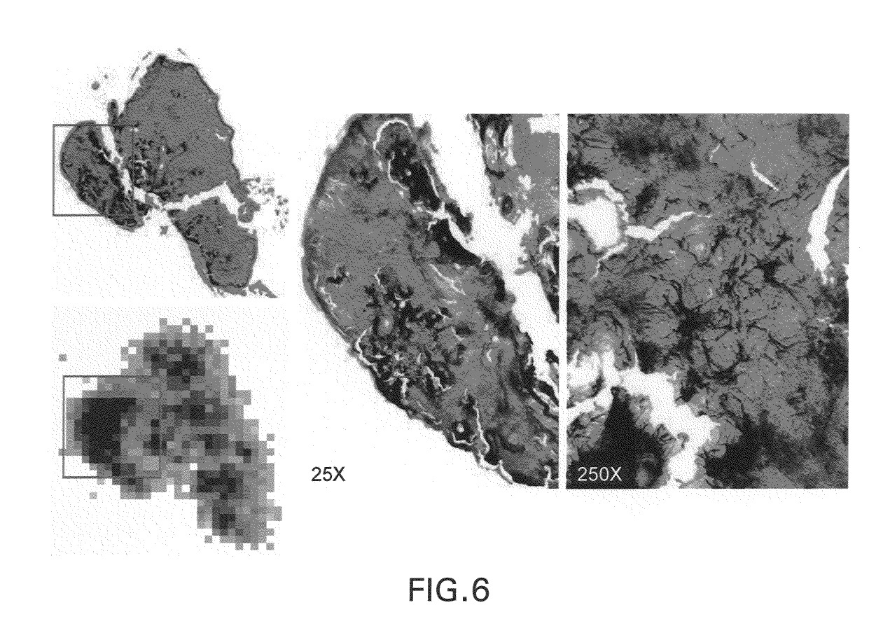

To confirm that tissue labeled with the imaging agent was actually infected with Aspergillus, histology of lung tissue labeled by the imaging agent in an infected mouse was performed. The corresponding gamma scintogram and histology data are show in FIG. 6. Aspergillus was demonstrated with the Grocott methenamine-silver nitrate (GMS) fungus staining technique. Note the black-stained organisms correlated with distribution of radioactivity in autoradiography (lower left image) of excised lung tissue from a mouse injected with Ga-68-labeled peptide of FIG. 4. See FIG. 6.

While embodiments of this disclosure have been depicted, described, and are defined by reference to specific example embodiments of the disclosure, such references do not imply a limitation on the disclosure, and no such limitation is to be inferred. The subject matter disclosed is capable of considerable modification, alteration, and equivalents in form and function, as will occur to those ordinarily skilled in the pertinent art and having the benefit of this disclosure. The depicted and described embodiments of this disclosure are examples only, and are not exhaustive of the scope of the disclosure.

1. An imaging composition comprising:
a fungus-specific peptide comprising at least a portion having the sequence of CGGRLGPFC (SEQ. ID. NO:1) or CWGHSRDEC (SEQ. ID. NO:2); and
an imaging material.

2. A composition according to claim 1, wherein the peptide comprises cyclic CGGRLGPFC (SEQ. ID. NO:1) or cyclic CWGHSRDEC (SEQ. ID. NO:2).

3. A composition according to claim 1, wherein the imaging material comprises a radionuclide.

4. A composition according to claim 1, further comprising a chelator.

5. A composition according to claim 1, further comprising a linker molecule.

6. A composition according to claim 1, further comprising a polymer.

7. A composition according to claim 1, further comprising a therapeutic agent.

8. A composition according to claim 1, further comprising aminobenzyl-DTPA and succinic acid, and wherein the peptide comprises cyclic CGGRLGPFC (SEQ. ID. NO:1) and the imaging material comprises ¹¹¹In.

9. A composition according to claim 1, wherein the wherein the peptide comprises cyclic CGGRLGPFC (SEQ. ID. NO:1) and the imaging material comprises ⁶⁸Ga, further comprising DOTA.

10. An imaging composition comprising:
a fungus-specific peptide comprising at least a portion having the sequence of CGGRLGPFC (SEQ. ID. NO:1) or CWGHSRDEC (SEQ. ID. NO:2); and
a chelator able to chelate a radionuclide.

11. A composition according to claim 10, wherein the peptide comprises cyclic CGGRLGPFC (SEQ. ID. NO:1) or cyclic CWGHSRDEC (SEQ. ID. NO:2).

12. A composition according to claim 10, further comprising a linker molecule.

13. A composition according to claim 10, further comprising a polymer.

14. A composition according to claim 10, further comprising a therapeutic agent.

15. A composition according to claim 10, wherein the peptide comprises cyclic CGGRLGPFC (SEQ. ID. NO:1) the chelator comprises aminobenzyl-DTPA, and the radionuclide comprises ¹¹¹In, further comprising succinic acid.

16. A composition according to claim 10, wherein the wherein the peptide comprises cyclic CGGRLGPFC (SEQ. ID. NO:1), the chelator comprises DOTA, and the imaging material comprises ⁶⁸Ga.

17. A method of detecting a fungal infection comprising:
administering an imaging agent to a patient, wherein the imaging agent comprises a fungus-specific peptide comprising at least a portion having the sequence of CGGRLGPFC (SEQ. ID. NO:1) or CWGHSRDEC (SEQ. ID. NO:2) and an imaging material; and
detecting the imaging agent in the patient, wherein detecting retained imaging agent in a tissue or organ indicates fungal infection of the tissue or organ.

18. A method according to claim 17, wherein the infection is an Aspergillus or Rhizopus infection.

19. A method according to claim 17, wherein the infection is in a lung of the patient.

20. A method according to claim 17, wherein detection includes gamma scintography or μ-PET.


SEQUENCE LISTING (2 sequences)

SEQ ID NO: 1 - protein, 9 aa, Artificial Sequence
  Description of Artificial Sequence Synthetic peptide
CGGRLGPFC

SEQ ID NO: 2 - protein, 9 aa, Artificial Sequence
  Description of Artificial Sequence Synthetic peptide
CWGHSRDEC